(19) United States
(12) Patent Application Publication
(10) Pub. No.: US 2024/0081887 A1
(43) Pub. Date: Mar. 14, 2024

(54) TIP PORTION FOR A CAUTERY DEVICE AND METHOD OF USE

(71) Applicant: ATEX TECHNOLOGIES, INC., Pinebluff, NC (US)

(72) Inventors: Joseph M. Shepley, Wernersville, PA (US); Brian McMurray, Aberdeen, NC (US)

(21) Appl. No.: 18/459,494

(22) Filed: Sep. 1, 2023

Related U.S. Application Data
(60) Provisional application No. 63/402,976, filed on Sep. 1, 2022.

(51) Int. Cl. A61B 18/08

(52) U.S. Cl. CPC A61B 18/082

(57) ABSTRACT

A tip portion for a cautery device defining a device axis is provided herein. The tip portion comprises a first electrode and a second electrode spaced apart from one another. The tip portion further comprises at least one wire comprising a first end in electrical connection with the first electrode, a second end in electrical connection with the second electrode, and a central portion in electrical communication with the first portion and the second portion. The central portion of the at least one wire defines a cautery shape comprising a continuous perimeter extending in a cautery plane, wherein the cautery plane is perpendicular to the device axis. A retrieval portion may be provided to retain a cut portion produced during use of the cautery device.

### CROSS-REFERENCE TO RELATED APPLICATION(S)

This application claims priority to U.S. Provisional Application No. 63/402,976, entitled “Tip Portion for a Cautery Device and Method of Use”, filed Sep. 1, 2022; the contents of which is hereby incorporated by reference in its entirety.

### FIELD OF THE INVENTION

Embodiments of the present invention relate generally to cautery devices and more specifically to tip portions for cautery devices used to create specifically sized and/or uniform cautery holes, such as in grafts, including, for example, vascular grafts, Valsalva grafts, or endo grafts.

### BACKGROUND OF THE INVENTION

Cautery devices are medical devices configured to conduct heat to create holes in mediums, for example, medical grafts and fabrics, skin, blood vessels, organs, and other objects.

Often cautery devices are configured as single-use handheld devices including one or more portable power source (e.g., batteries). A typical cauterizing instrument comprises a heating element in electrical communication with a power source (e.g., one or more batteries), a switch for selectively completing the power circuit between the heating element and the power source, and a housing for enclosing the power source and associated electrical connections. The housing for enclosing the power source is configured to indicate the power capability of the housing, for example, a smaller housing may only include a single battery, while a larger housing may hold more than one battery, and thus, the larger housing indicates a larger power output capability. The heating element may be configured as a metal tip, such that electrical power from the power source may cause the metal tip to heat up. In this regard, a smaller housing produces a lower maximum temperature of the metal tip, and a larger housing produces a greater maximum temperature of the metal tip. A user (e.g., medical professional) may move the metal tip into contact with the surface being cut and operate the cauterizing instrument to create a hole therein. During use, the heated metal tip may reach very high temperatures, (e.g., in excess of 2,200° F.), which may cause the medium to char, burn, and/or ignite. This is particularly true because a medical professional may have to manipulate or steer the metal tip to form a desired hole shape—leading to an increased opportunity of too much time for the metal tip to overheat and burn, char, or ignite the surface—particularly when dealing with grafts. Additionally, the small housing and the large housing comprise electrical connections such that the metal tip may be inserted into either the small housing or the large housing. In this regard, a user may insert the metal tip into housing with an incorrect power supply thereby causing burning and/or charring when the power is too high, or increasing the melting time when the power is too low.

In many cases too, the portion of the surface being removed may fall into the surgical area, such as into the remainder of the graft. This extra portion of graft can be problematic for removal/retrieval and lead to unwanted contamination, embolization, further injury, a wasted graft, extra time, among other issues.

### BRIEF SUMMARY OF THE INVENTION

Embodiments of the present invention are directed towards cautery devices and corresponding tip portions for cautery devices used to create holes in a medium (e.g., medical grafts, fabrics, skin, blood vessels, organs, tissue, and other objects). The tip portion may comprise at least one wire in electrical communication with electrodes. The at least one wire may be shaped into a cautery shape, corresponding to the perimeter of the hole to be made. The cautery shapes may be closed shapes (e.g., circles, ovals, diamonds, etc.) of varying sizes. In some embodiments, different shapes and/or sizes may be color coded, such as to provide for ease of selection and/or visual confirmation (which may be helpful during a procedure). Having a continuous perimeter of shape in the plane that is performing the cut helps ensure an accurate cut and prevents the need for a medical professional to “move around” a metal tip.

In some embodiments, the cautery tip portion may comprise a retrieval portion, (e.g., a needle, hook, etc.) that may be configured with a rough surface (e.g., barbs, divots, or other protrusions) that are configured to interact with and retain the medium during and after the application of heat to the cut the medium. In some embodiments, other types of retrieval portions are contemplated, such as suction-based retrieval portions (e.g., utilizing vacuum suction, etc.). Notably, the portion of the medium corresponding to the cautery shape is maintained on the retrieval portion when the cautery device is removed from the medium. Said differently the retrieval portion may retain the portion of the medium corresponding to the cautery shape on the body of the retrieval portion after the tip portion is removed from the medium. Additionally, in some embodiments, the tip portion may be used to remove multiple portions of the medium during the same operation, in this instance the portions of the medium may be stacked on the retrieval portion body (or removed between each use). This helps remove the cut portion from the surgical area safely without retrieval or using extra equipment or additional personnel to “hold” a portion as it is cut. In some embodiments, a retrieval portion may be provided separately from a cutting mechanism, such as may be used with a separate hand or by another medical professional. By utilizing a separate device, additional angles may be achieved, which may be beneficial depending on the site being worked on.

Some embodiments of the present invention utilize high resistance alloys for the at least one wire such that the at least one wire may heat to a temperature high enough to melt the medium, while maintaining the temperature of the wire below a burning temperature of the medium.

In an example embodiment a tip portion for a cautery device defining a device axis is provided. The tip portion comprises a first electrode and a second electrode spaced apart from one another. The tip portion further comprises at least one wire. The at least one wire comprising a first end in electrical connection with the first electrode, a second end in electrical connection with the second electrode and a central portion in electrical communication with the first electrode and the second electrode. The central portion defines a cautery shape. The cautery shape comprises a continuous perimeter extending in a cautery plane. The cautery plane is positioned at an angle from the device axis.

In some embodiments, the tip portion may further comprise a retrieval portion positioned between the first electrode and the second electrode. In some embodiments, the retrieval portion may extend along and may be moveable along a retrieval axis.

In some embodiments, the tip portion may further comprise a stabilizer. In some embodiments, the first electrode, the second electrode and the retrieval portion may extend through the stabilizer. The stabilizer may be configured to maintain spacing between the first electrode and the second electrode.

In some embodiments, the tip portion may further comprise at least one shuttle connected to the retrieval portion and moveable along the retrieval axis to enable movement of the retrieval portion between a retracted position and an extended position. In some embodiments, when the retrieval portion is in the extended position a tip of the retrieval portion extends past the cautery plane. In some embodiments, when the retrieval portion is in the extended position the top of the retrieval portion extends though the continuous perimeter of the cautery shape. In some embodiments, the tip portion may further comprise at least one biasing element configured to bias the needle to the retracted position. In some embodiments, when the retrieval portion extends through the cautery plane, the retrieval portion may be configured to pass through a medium being cut. The bias of the biasing element may cause the retrieval portion to pull the medium towards the at least one wire.

In some embodiments, upon application of electricity to the at least one wire, the at least one wire may cause cutting of a hole in the medium in the cautery plane. Upon completion of the cutting of the hole, tension from the medium holding the retrieval portion against the bias of the biasing element is removed to enable the retrieval portion and a corresponding portion of the medium attached thereto to move to the retracted position such that the portion of the medium being cut is retained by the retrieval portion.

In some embodiments, the retrieval portion may be barbed. In some embodiments, the retrieval portion may be a needle. In some embodiments, the at least one wire may be made from an iron-chromium-aluminum alloy or a nickel-chromium alloy. In some embodiments, the perimeter of the cautery shape may define a variety of shapes, e.g., circle, oval, diamond, etc. In some embodiments, the first electrode and the second electrode may be configured to cause the wire to heat to a temperature between 700 F-2200 F. In some embodiments, the temperature may be below a burning temperature of a vascular graft. In some embodiments, the cautery plane may be at least 3 mm from an end of the first electrode and an end of the second electrode.

In some embodiments, the cautery device may comprise a first receiving hole and a second receiving hole. The first electrode may be configured to be received by the first receiving hole, and the second electrode may be configured to be received by the second receiving hole.

In another embodiment a cautery tip portion for a cautery device, defining a device axis, is provided. The cautery tip portion comprises a first electrode and a second electrode spaced apart from one another. The cautery tip portion further comprises at least one wire. The at least one wire is in electrical connection with the first electrode and the second electrode. The at least one wire defines a cautery shape. The cautery shape comprises at least a near-continuous perimeter extending in a cautery plane. The cautery plane is positioned at an angle to the first electrode and the second electrode. The cautery tip portion further comprises a retrieval portion disposed between the first electrode and the second electrode. The retrieval portion extending along and being movable along a retrieval axis.

In some embodiments, the cautery tip portion may further comprise a stabilizer. The first electrode, the second electrode and the needle may extend through the stabilizer. In some embodiments, the cautery tip portion may further comprise at least one shuttle connected to the retrieval portion and moveable along the retrieval axis to enable movement of the retrieval portion between a retracted position and an extended position. In some embodiments, in the extended position a tip of the retrieval portion may extend past the cautery plane. In some embodiments, the cautery tip portion may further comprise at least one biasing element configured to bias the retrieval portion to the retracted position. In some embodiments, the retrieval portion may be barbed.

In yet another embodiment a method of creating a hole using a cautery device is provided. The method comprises providing a cautery device defining a device axis, comprising a cautery tip portion. The cautery tip portion comprises a first electrode and a second electrode extending through a stabilizer, configured to maintain spacing between the first electrode and the second electrode. The cautery tip portion further comprises at least one wire. The at least one wire is in electrical connection with the first electrode and the second electrode. The at least one wire defines a cautery shape. The cautery shape comprises at least a near-continuous perimeter extending in a cautery plane. The cautery plane is positioned at an angle to the first electrode and the second electrode. The cautery tip portion further comprises a retrieval portion disposed between the first electrode and the second electrode. The retrieval portion extending along and being movable along a needle axis. The cautery tip portion further comprises at least one shuttle connected to the retrieval portion and moveable along the needle axis to enable movement of the retrieval portion between a retracted position and an extended position. The cautery tip portion further comprises at least one biasing element configured to bias the retrieval portion to the retracted position. The method further comprises puncturing a graft with the retrieval portion, such that the graft is in contact with the at least one wire. The retrieval portion providing tension on the graft. The method further comprises cutting the graft in the cautery shape. The first electrode and the second electrode are configured to heat the at least one wire to a temperature, wherein the temperature is configured to cut the graft. The method further comprises retaining a cautery shaped portion of the graft on the retrieval portion after the graft is cut.

### BRIEF DESCRIPTION OF THE DRAWINGS

Having thus described the invention in general terms, reference will now be made to the accompanying drawings, which are not necessarily drawn to scale, and wherein:

FIG. 1 illustrates an example tip portion for a cautery device, in accordance with some embodiments discussed herein;

FIG. 2A illustrates a close up view of a first portion of the example tip portion shown in FIG. 1, with a retrieval portion shown in a retracted position, in accordance with some embodiments discussed herein;

FIG. 2B illustrates the first portion of the example tip portion shown in FIG. 1, with the retrieval portion shown in an extended position, in accordance with some embodiments discussed herein;

FIG. 3 illustrates a close-up view of a second portion of the example tip portion shown in FIG. 1, in accordance with some embodiments discussed herein;

FIGS. 4A-D illustrate a variety of example wire configurations, defining different cautery shapes or sizes, in accordance with some embodiments discussed herein;

FIGS. 5A-C illustrate further example cautery shapes extending within a cautery plane, in accordance with some embodiments discussed herein;

FIG. 6 illustrates example cautery devices including the example cautery tip portion, shown in FIG. 1, in accordance with some embodiments discussed herein;

FIGS. 7A-7E illustrate an example cautery device forming a hole in an example graft, in accordance with some embodiments discussed herein; and

FIG. 8 illustrates a flow chart of an example method of using a cautery device to form a hole in a graft, in accordance with some embodiments discussed herein.

### DETAILED DESCRIPTION OF THE INVENTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout.

Cautery devices utilize heat to remove or seal a medium, sometimes burning and/or destroying the medium. Some cautery devices are used to create one or more holes in mediums, including tissue, grafts, synthetic grafts, medical fabrics, or other objects. Notably, the grafts may be designed for use with attachment to blood vessels or organs within a patient. In some cases, the graft may already be installed within the patient when the cut is needed to be made, although the cautery device may be used prior to insertion of the graft within the patent (e.g., the cautery device may be used to make a branched graft—which may need to be customized during surgery based on the anatomy of the patient). While some embodiments of the present invention described herein reference vascular grafts, embodiments of the present invention may be used with other grafts, such as endo grafts. Additionally, embodiments of the present invention may be used with other surfaces/objects.

As discussed, formation of cautery holes are typically free handed, as a user would move the cautery device around on the surface as the cutting (e.g., melting) occurs to create a desired shape for the one or more holes. In the regard the hole(s) created were not uniform, may comprise rough edges, may be misshaped and may lack a consistent size and/or shape between multiple holes. Additionally, the wire may span a temperature range where the wire is configured to exceed a melting temperature of the medium, and may cause the medium to char, burn, and/or in some instances cause the medium to ignite. Along these lines, in some cases, too much time was spent on locations and charring, burning, etc. occurred. Further, different cautery device sizes may be provided that have different temperature ranges, meaning that a medical professional may grab the wrong one, which may have too high of a temperature—thereby causing charring or burning (or igniting) of the surface to occur. Further, the medium corresponding to the hole(s) were required to be removed separately. All of these are variables, when operating under time constraints of surgery (e.g., replacing arteries around the heart) are undesirable, as there is a proscribed amount of time to complete the procedure. Thus, a cautery device which reduces and/or controls these variables is desirably provided herein. For example, some embodiments of the present invention provide a cautery device and tip portion that is configured to retain the cut portion of the medium, create a uniform hole, melt but not burn the medium, and provide consistent shapes and sizes to holes formed with the same tip portion. Since the tip portion corresponds to the hole to be formed, the user may assess the cautery shape of the tip portion prior to the procedure, such as to plan the procedure.

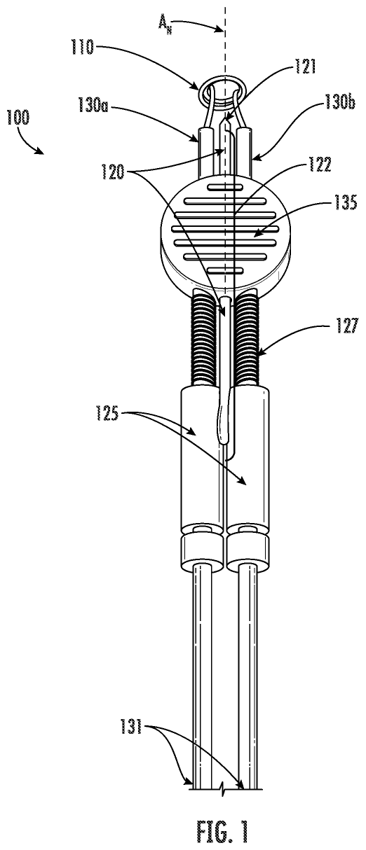

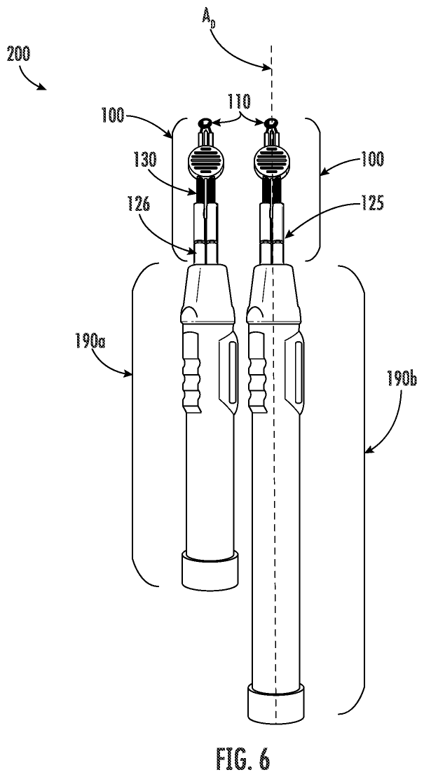

FIG. 1 illustrates an example tip portion 100 for a cautery device (see e.g., cautery device(s) 200, FIG. 6). The tip portion 100 comprises a first electrode 130a and a second electrode 130b (also referred to as “the electrodes 130”) spaced apart from one another. In some embodiments, the first electrode 130a and the second electrode 130b may be parallel, while in other embodiments, the first electrode 130a and the second electrode 130b may be angled towards one another or away from one another. In some embodiments, an end side 131 of each of the first electrode 130a and the second electrode 130b may be configured to be received by the cautery device housing (e.g., 190, FIG. 6) such that the cautery device housing may provide electrical current through the first electrode 130a and the second electrode 130b.

The tip portion 100 may comprise at least one wire 110 in electrical connection with the first electrode 130a and the second electrode 130b. The at least one wire 110 may define a cautery shape, wherein the cautery shape defines the dimensions of the hole to be formed in the medium (e.g., medical grafts, fabrics, skin, blood vessels, organs, and other objects or implants). In some embodiments, though not illustrated, multiple wires may be used to form the at least one wire and/or cautery shape.

The tip portion 100 may further comprise a stabilizer 135. Each of the first electrode 130a and the second electrode 130b may be positioned through the stabilizer 135, such that the stabilizer 135 maintains the spacing between the first electrode 130a and the second electrode 130b.

In some embodiments, the tip portion 100 may additionally comprise a retrieval portion 120 extending along a retrieval axis A_{N}. The needle 120 may define a retrieval body 122 and a tip 121 extending from the retrieval body 122. In some embodiments, the retrieval axis A_{N} may extend parallel to the first electrode 130a and the second electrode 130b through the stabilizer 135. In this regard, the retrieval portion 120 may be disposed between the first electrode 130a and the second electrode 130b and positioned through the stabilizer 135. In some embodiments, the retrieval portion 120 is configured to be moveable along the retrieval axis A_{N}. Notably, the retrieval portion and the retrieval axis, in some embodiments, may be directed at a different angle with respect to various components of the tip portion 100 (e.g., angled through the stabilizer 135, not parallel with the electrodes, etc.).

In some embodiments, the retrieval portion 120 defines a retrieval length. In some embodiments, the retrieval length may the length of the body 122, as the size of the tip 121 may be de minimus. In some embodiments, the size of the tip 121 may be taken into account in the retrieval length. In some embodiments, the retrieval length may be at least 30 mm, at least 40 mm, or at least 50 mm long. In some embodiments, the retrieval portion may be 42 mm long. In some embodiments, the retrieval portion 120 may be a needle, as illustrated, a hook, a holder, a gripper, a pin, or similar, providing the retrieval portion 120 terminates in a tip 121 which may penetrate a graft or other medical mesh. In some embodiments, the retrieval portion 120 may be stainless steel, titanium, or other suitable metal.

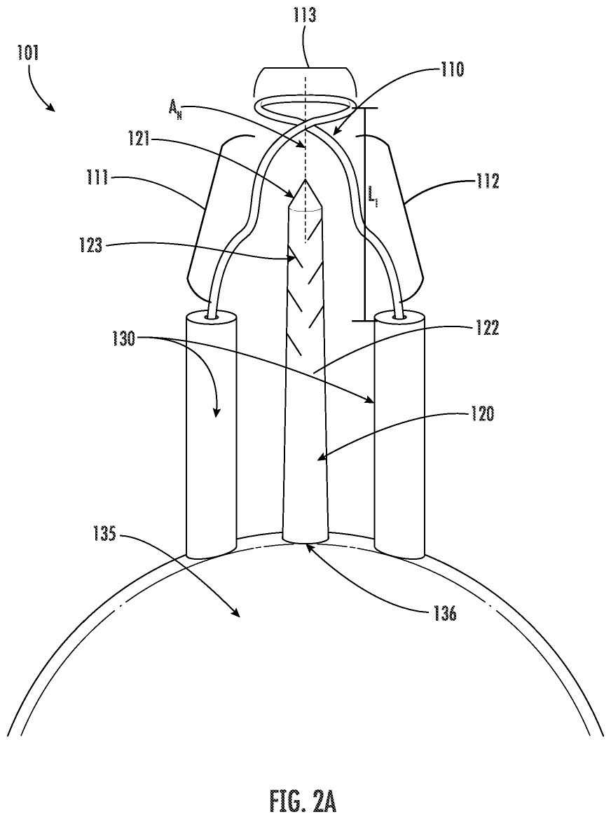

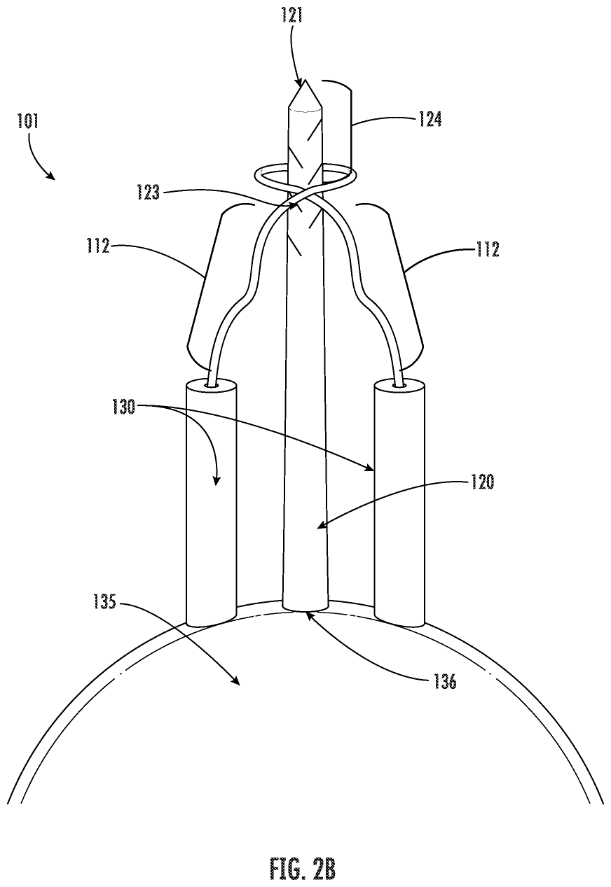

The tip portion 100 may further comprise at least one shuttle 125 connected to the retrieval portion 120. The at least one shuttle 125 may be positioned about at least one of the first electrode 130a or the second electrode 130b such that the at least one shuttle 125 is moveable about the respective electrode(s) parallel to or along the retrieval axis A_{N}. In this regard the at least one shuttle 125 may facilitate movement of the retrieval portion 120 between a retracted position, illustrated in FIG. 2A, and an extended position, illustrated in FIG. 2B. In some embodiments, the retrieval axis A_{N} may be at an angle (e.g., a non-zero angle) in relation to the surface being cut (e.g., a graft). In some such embodiments, the retrieval axis A_{N} may be at a corresponding angle (e.g., a non-zero angle) with respect to a cautery (e.g., cutting) plane corresponding to, for example, the at least one wire 110. In some example embodiments, the retrieval axis A_{N} may extend at an angle with respect to the surface being cut and/or the at least one wire 110, where the angle ranges from 1 degree to 85 degrees, such as about 35 degrees, about 40 degrees, about 45 degrees, or about 50 degrees, etc.

In some embodiments, the tip portion 100 may comprise at least one biasing element 127. The at least one biasing element 127 may be positioned between the at least one shuttle 125 and the stabilizer 135 about the respective one of the electrodes 130. The biasing element 127 may be configured to bias the needle 120 in the retracted position. In the illustrated embodiment, the at least one biasing element 127 comprises two springs, each spring being positioned about a respective electrode between the stabilizer 135 and the shuttle 125.

FIGS. 2A-B illustrate a first portion 101 of the tip portion 100, with the retrieval portion 120 in a retracted position and an extended position, respectively.

As discussed above, each of the electrodes 130 may be in electrical communication with the at least one wire 110. The electrodes 130 may be formed of brass in some embodiments, but other suitable conductive metals may be used. In some embodiments, the electrodes 130 may be a wrapped wire, wherein the wire is a conductive metal, for example copper. In some embodiments, the wrapping of the wire may be color coded to indicate to the user, for example, the available current density for the tip portion 100. In some embodiments, the electrodes 130 may be the same length and thickness. In some embodiments, the electrodes 130, such as when coupled with the cautery device housing, may be in electrical communication with a power source (e.g., one or more batteries) to receive power for causing the at least one wire 110 to heat up. In some embodiments, the size (e.g., length and shape) and composition of the electrodes 130 may be customized to the medium used. In this regard, changing the size of the electrode may change the current density applied through the at least one wire 110. Thus, the size and composition of the electrodes 130 may be customized to the medium to allow formation of a cautery hole within the medium, without burning, charring, or igniting the surrounding medium.

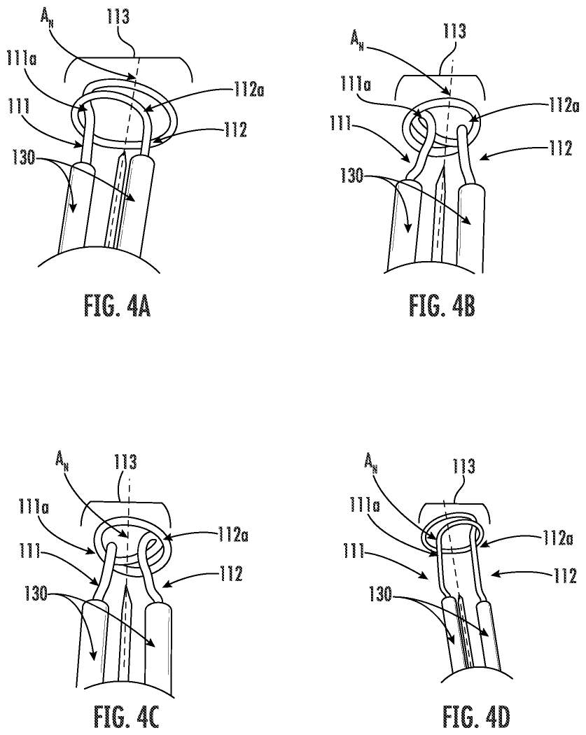

As mentioned, the at least one wire 110 may be in electrical communication with the electrodes 130. More particularly, in some embodiments, the at least one wire 110 may comprise a first portion 111, a second portion 112, and a central portion 113, wherein the central portion 113 extends between the first portion 111 and the second portion 112. In some embodiments, the first portion 111 may be connected directly with the first electrode 130a, and the second portion 112 may be connected directly with the second electrode 130b. In some embodiments, with reference to FIGS. 4A-B, the first portion 111 and the second portion 112 may extend linearly from the electrodes 130 to the central portion 113, while in other embodiments the first portion 111 and the second portion 112 may be angled towards the central portion 113. In this regard, in some embodiments, the first portion 111 and the second portion 112 may extend parallel to the retrieval axis A_{N}, while in other embodiments, the first portion 111 and the second portion 112 may extend from the electrodes 130 at an angle relative to the retrieval axis A_{N}.

Returning to FIG. 2A, in some embodiments, the first portion 111 and the second portion 112 may be non-linear. In this regard, the first portion 111 and the second portion 112 may comprise at least one bend. The at least one bend may provide stability to the at least one wire 110 during cauterization. In some embodiments, the first portion 111 and the second portion 112 may be angled so that a portion of each along its length may be contained within a footprint of the cautery shape of the central portion 113 (as described further herein)—which may aid in preventing accidental application of heat via such portions of the wire.

In some embodiments, the first portion 111 and the second portion 112 each define a length. The length may be configured to be minimize the amount of resistance through the at least one wire 110, and thus, require less power to heat the at least one wire 110 to the requisite temperature. In some embodiments, the first portion 111 and the second portion 112 may be less than 5 mm, less than 4 mm or even less than 3 mm long.

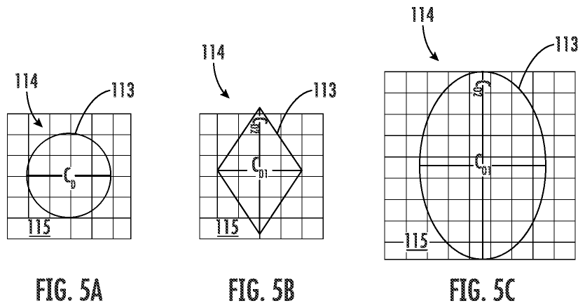

In some embodiments, rather than measure the length of the first portion 111 and the second portion 112, in some embodiments, a length L₁ may be measured between the connection of the electrodes 130 and the cautery plane (see e.g., 115, FIG. 5A). In this regard, in some embodiments, the length L₁ may be less than 5 mm, less than 4 mm, or less than 3 mm long. As discussed above, the length of the at least one wire correlates to the power required to heat the at least one wire to the requisite temperature. Thus, with a shorter length L₁ between the connection of the electrodes 130 and the cautery plane, the wire may apply a relatively larger amount of heat and/or heat up faster. In some embodiments, the at least one wire 110 may be an iron-chromium-aluminum alloy, for example Kanthal® or a nickel-chromium alloy (e.g., Nichrome), or another alloy with similar resistance heating characteristics. In some embodiments, the wire length L₁, and the composition of the at least one wire 110 may be correlated to the medium. In this regard, the cautery tip may be customized to the needed temperature profile by changing the wire length L₁ and/or the composition of the at least one wire 110, to create a cautery hole in the medium while preventing burning, charring, or ignition of the medium surrounding the cautery hole.

In some embodiments, the central portion 113 may extend between the first portion 111 and the second portion 112. In some embodiments, the central portion 113 may be formed with a center of a single wire—although, in some embodiments, multiple wires or wire portions may be used. In some embodiments, the central portion 113 may be formed into a cautery shape. With reference to FIGS. 4A-4D and 5A-5C, the cautery shape 114 may comprise a continuous (or near-continuous) perimeter created by the central portion 113 of the at least one wire 110. A perimeter may be near-continuous if there is one or more minor breaks in the perimeter, such as due to a spacing between separate portions of the wire bending to form the cautery shape. In some embodiments, the cautery shape 114, may extend within a cautery plane 115. To explain, the cautery plane 115 may extend along a plane that correlates to an intended cut of the medium. In some embodiments, the cautery plane 115 may be configured relative to the retrieval axis A_{N} such that the retrieval portion 120 may extend through the cautery plane 115. Thus, in some embodiments, the cautery plane 115 may be perpendicular to the retrieval axis A_{N} and/or device axis, while in other embodiments, the cautery plane 115 may be angled (e.g., not parallel) with respect to the retrieval axis A_{N} and/or device axis. In some embodiments, the cautery plane 115 may extend at an angle within the range of 450 to 900 relative to the retrieval axis A_{N} or device axis—although other angle ranges are contemplated (e.g., 55°-90°, 65°-90°, 75°-90°, 80°-90°, 85°-90°, 88°-90°, etc.).

Returning to FIG. 2A, in some embodiments, the tip 121 of the retrieval portion 120 may be configured to pierce the medium, for example a graft or a medical fabric. In this regard, in some embodiments, the surface of the tip 121 may be pointed and may be smooth such as to ease insertion into the medium. In contrast, at least a portion of the body 122 of the retrieval portion 120 may be rough, and in some embodiments, a portion of the body 122 near the tip 121 may comprise barbs 123. The barbs 123 may be configured as protrusions, divots, etc. configured to snag and/or catch the medium pierced by the tip 121. In some embodiments, the barbs 123 may be configured at an angle relative to the retrieval axis A_{N}, and in some embodiments the barbs 123 may be perpendicular to the retrieval axis A_{N}. In some embodiments, the barbs 123 may be parallel to one another, while in other embodiments the barbs 123 may be randomly spaced about the body 122 of the retrieval portion 120. In some embodiments, each of the barbs 123 may extend about the circumference of the body 122, while in other embodiments, each of the barbs 123 may extend about a portion of the circumference of the body 122. In some embodiments, the barbs 123 may be a single protrusion or divot extending about the circumference of the body 122 (e.g., in a spiral).

In some embodiments, the barbs 123 may cover the entire body 122 of the needle 120, while in other embodiments, the barbs 123 may cover a portion of the body 122 close to the tip 121, which may pass through the cautery plane 115 when the retrieval portion 120 is in the extended position, such as illustrated in FIG. 2B. Such a configuration with barbs 123 only positioned proximate the tip 121 may be helpful to minimize resistance or undesired snags within the stabilizer 135 as the retrieval portion 120 moves between the extended position and the retracted position. In some embodiments, the retrieval portion 120 may be configured for multiple retrievals. In this regard, the retrieval portion 120, including the barbs 123 may retain multiple portions of medium corresponding to the cautery shape. For example, when more than one hole is created by the same tip portion 100, the portions of the medium may be stacked on the body 122, or each portion of the medium may be removed from the body 122 of the retrieval portion 120 once the tip portion 100 is removed from the medium.

Accordingly, the retrieval portion 120 may be configured to pierce a medium with the tip 121 and retain the medium about the body 122 due the rough surface, or the barbs 123. In this regard as explained herein, the retrieval portion 120 may help to retain contact between the medium and the at least one wire 110 during cauterization and retain the cut portion of the medium after cauterization.

In some embodiments, the needle 120 may be positioned through the stabilizer 135 via a hole 136. In some embodiments, the hole 136 may be centered between the electrodes 130. In this regard, the retrieval portion 120 may be configured to move through the stabilizer 135 along the retrieval axis A_{N} such that the retrieval extends through the cautery shape 114, and the cautery plane 115 when transitioned into the extended position, such as illustrated in FIG. 2B. This may be accomplished by a medical professional moving the shuttle 125 along the retrieval axis A_{N} toward the medium to be cut. Such movement may occur against the bias of the biasing element(s) 127.

In the extended position, with reference to FIG. 2B, a portion 124 of the retrieval portion 120 including the tip 121 and a segment of the body 122 are positioned through the cautery plane. In some embodiments, the retrieval portion 120 may be positioned in the center of the cautery shape 114, while in other embodiments, the retrieval portion 120 may be off center of the cautery shape 114.

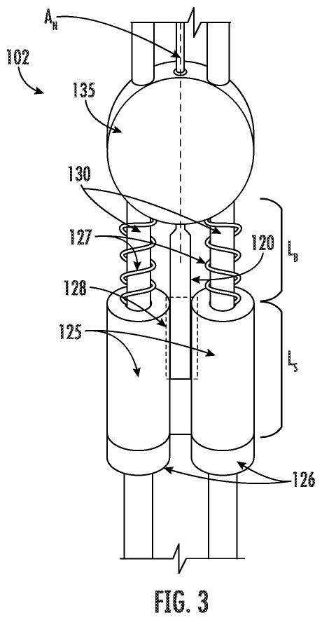

FIG. 3 illustrates a second portion 102 of the insert (which is positioned below the first portion 101). The second portion 102 includes the shuttle 125. While illustrated as a single component (with sections—one for each electrode), in some embodiments, the at least one shuttle 125 may be a first shuttle and a second shuttle. In this regard, the first shuttle may be moveable along the first electrode 130a and the second shuttle may be movable along the second electrode 130b. In some embodiments, the at least one shuttle 125 may define a shuttle length Ls. The shuttle length Ls may be between 5-25 mm, between 10-20 mm, and in some embodiments may be between 12-17 mm. The at least one shuttle 125 may be positioned about one of the electrodes 130.

In some embodiments, the at least one shuttle 125 may be moveable along one of the corresponding electrodes 130 between a shuttle end stop 126 and the stabilizer 135. In some embodiments, the shuttle end stop 126 may be positioned along one of the corresponding electrodes 130 such that the shuttle end stop 126 abuts a housing of the cautery device, when the electrodes 130 are positioned within respective openings within the housing of the cautery device.

As discussed above, the at least one shuttle 125 may be connected to the retrieval portion 120 at a connection position 128. In some embodiments, the retrieval portion 120 may be connected to the at least one shuttle 125 via an adhesive, for example a glue, or a tape. In other embodiments, the retrieval portion 120 may be connected to the at least one shuttle via an interference fit, or by fusing the retrieval portion 120 to the at least one shuttle 120. In some embodiments, at least 10 mm of the retrieval portion 120 may be attached to the at least one shuttle 125 at the connection position 128. While in some embodiments, the connection portion may comprise at least 5 mm, and no more than 15 mm of the needle 120.

In some embodiments, at least one biasing element 127 may be positioned between the at least one shuttle 125 and the stabilizer 135 on the corresponding one of the electrodes 130. In some embodiments, the at least one shuttle 125 may be configured to move from a biased positioned to an unbiased position. In some embodiments, in the biased position the at least one shuttle 125 may abut the end stop 126, and, in the unbiased position, the at least one shuttle 125 may abut the stabilizer 135. In this regard, the at least one biasing element 127 may bias the retrieval portion 120 to the retracted position. Thus, to move the needle 120 from the retracted position to the extended position, a force may be applied to the at least one shuttle 125, thereby overcoming the bias force of the biasing element 127.

In some embodiments, the at least one biasing element 127 may be a spring. The spring may be wound around the electrode corresponding to the at least one shuttle 125. In some embodiments, when utilizing a single spring as the biasing element 127, the spring may define a spring constant of 0.005 N/m. In another example embodiment, the at least one biasing element 127 may be two springs. The first spring may be wound about the first electrode 130a and the second spring may be wound around the second electrode 130b. In this regard, the spring constant of each of the first and second springs may be lower than if there was a single spring, as both springs contribute to the biasing of the at least one shuttle 125 and the retrieval portion 120. Thus, in the example embodiment, the first and second spring may define a spring constant of less than 0.005 N/m, for example 0.0045 N/m.

In some embodiments, the at least one biasing element 127 may define a biasing element length L_{B}. In some embodiments, the biasing element length L_{B} may be sized to allow the retrieval portion 120 to sufficiently pierce the medium, such that the barbs 123 positioned on the body 122 engage the medium. In this regard, the may biasing element length L_{B} be greater than a distance between the tip 121 of the retrieval portion 120 and the cautery plane 115 when the retrieval portion 120 is in the retracted position. Therefore, in the extended position the retrieval portion 120 may be able to extend though the cautery plane. In some embodiments, the shuttle length L_{S} may be longer than the biasing element length L_{B}. In some embodiments, the biasing element length L_{B} may be less than 15 mm. In some embodiments, the biasing element length L_{B} may be about 10 mm.

As discussed, the at least one wire 110 may be formed in different configurations thereby defining a variety of shapes and sizes for the cautery shape—which enables specialization for different cautery procedures. FIGS. 4A-D illustrate different configurations of the first end 111, the second end 112, and the central portion 113 of the at least one wire 110, where each configuration defines a circular cautery shape within the cautery plane. Notably, each cautery shape defines a continuous or near-continuous perimeter.

To explain, in FIG. 4A, the first end 111 and the second end 112 extend parallel to the needle axis A_{N} between the electrodes 130 and the central potion 113. Thus, the central portion 113 of the at least one wire 110 comprises a section between the electrodes 130 wherein the at least one wire 110 overlaps on itself. To form a continuous perimeter, a first transition point 111a between the first end 111 and the central portion 113 is positioned interior to the cautery shape. Thus, when the cautery shape contacts the medium, the transition point 111a allows the central portion 113 to create a continuous perimeter for fulling cutting the medium. Similarly, a second transition point 112a between the second portion 112 and the central portion 113 is below but follows the perimeter of the cautery shape. Thus, a portion of the central portion 113 may be configured to not engage with the medium. In this regard, the central portion 113 may be configured to be a continuous perimeter. In the illustrated embodiment, the diameter of the cautery shape (e.g., the footprint) may be similar to the space between the electrodes 130.

In some embodiments, the configuration of the first portion 111 and the second portion 112, such as illustrated in FIG. 4B, may result in a smaller cautery diameter (than, for example, in FIG. 4A). As illustrated, the first portion 111 and the second portion 112 are configured at an angle compared to the retrieval axis A_{N}. In this regard, both transition points 111a, 112a between the first portion 111 and the central portion 113 and the second portion 112 and the central portion 113 are interior to the cautery shape, thus, there may be a lower risk of melting the medium positioned external to the cautery shape.

In some embodiments, such as illustrated in FIG. 4C, the first end 111 and the second end 112 are angled toward from the retrieval axis A_{N} at the connection point between the first end 111 and the second end 112 and the respective electrode 130. In some embodiments, each of the first end 111 and the second end 112 may transition to be parallel with the retrieval axis A_{N}. In this regard, the configuration of the first and second transition points 111a, 112a between the first end 111 and the central portion 113 and the second end 112 and the central portion 113 may be similar to those described with reference to FIG. 4A.

In some embodiments, such as illustrated in FIG. 4D, the first end 111 and the second end 112 may be angled from the retrieval axis A_{N}. Rather than be angled toward the retrieval axis A_{N}, as illustrated in FIGS. 4B-C, the first end 111 and the second end 112 may be angled backwards such that the first and second transition points 111a, 112a between the first end 111 and the central portion 113 and the second end 112 and the central portion 113 may abut, or almost abut. In this regard, rather than the retrieval portion 120 extending between the first end 111 and the second end 112, the needle 120 may extend in a different axis.

With reference to FIGS. 5A-C, the at least one wire 110 may define a variety of cautery shapes 114. While such shapes are shown as continuous perimeters, near-continuous perimeters may be used—as described herein. In some embodiments, such as illustrated in FIG. 5A, the cautery shape 114 may be a circle defining a cautery diameter C_{D} extending across within a cautery plane 115. In some embodiments, the cautery diameter C_{D} may be up to 2 mm, up to 3 mm, or up to 5 mm. In some embodiments, the cautery diameter C_{D} may be less than 5 mm, less than 3 mm, or even less than 2 mm. In some embodiments, the cautery diameter C_{D} may be sized to the cautery hole to be formed. In this regard, the cautery diameter C_{D} may be greater than 5 mm, for example, in some embodiments the cautery diameter C_{D} may be at least 5 mm, at least 7 mm, or even greater than 10 mm. In some embodiments, the cautery diameter C_{D} may be customizable for the use of the cautery tip.

In some embodiments, the cautery shape may be a diamond, such as illustrated in FIG. 5B (although a square or rectangle is also contemplated). In some embodiments, a diamond cautery shape may be used for cutting through stents, for example, when the cross section structure of a stent is a diamond shape. Thus, the cautery shape of a diamond enables cutting between the cross section structure of the stent.

Similarly, in some embodiments, the cautery shape may be an oval, such as illustrated in FIG. 5C. An oval cautery shape may be used to assemble grafts, or for example in remodeling a graft. When using an insert with an ovate, or a diamond shaped cautery shape, it may be the cautery shape must have two correct measurements, a first diameter C_{D1}, C_{O1} and a second diameter C_{D2}, C_{O2}. The two diameters may inform the user of how the cautery hole will be formed. In some embodiments, the at least one wire 110 may be formed into customizable shapes, for example, the shape, size, and/or dimensions may be customized for the specific procedure.

With reference to FIG. 3, in some embodiments, the stabilizer 135 may present an indication of the cautery shape, cautery size, and/or current density of the electrodes. In this regard, the stabilizer 135 may be printed on, color-coded, or otherwise include or provide indicia for the user to visually see the specifications of the tip portion 100.

As discussed above, and with reference to FIG. 6, a cautery device 200 may include the tip portion 100 and a cautery device housing 190. FIG. 6 illustrates two example cautery devices, where the cautery device housing on the left 190a holds a single AA battery, while the cautery device housing on the right 190b holds two AA batteries. The tip portion 100 may be positioned in a cautery device housing 190, such as either cautery device housing 190a, 190b. The cautery device housing 190a, 190b, may define a device axis A_{D}. In some embodiments, the device axis A_{D} may be an extension of the retrieval axis A_{N}. In some embodiments, the cautery device housing 190 may comprise a power source, and a power switch 191. In some embodiments, the power source may be at least one battery. In some embodiments, the size of the cautery device housing 190 may correspond to the quantity of batteries disposed within the cautery device housing 190. For example, a cautery device housing 190a may be configured to receive a single battery, while a second cautery device housing 190b may be configured to receive two batteries. In some embodiments, the number of batteries positioned within the cautery device housing 190 and/or their power level(s) may determine the amount of time for the at least one wire 110 to reach the desired temperature. In some embodiments, the configuration of the electrodes 130 (e.g., shape and/or composition) and the configuration of the at least one wire 110 (e.g., composition and/or length) in addition to the power output of the cautery device housing 190 may determine the amount of time for the at least one wire 110 to reach the desired temperature, and the maximum temperature of the at least one wire 110. In this regard, a benefit of some embodiments of the present invention include the ability for the cautery tip to be used with either a “low” power or a “high” power cautery device without causing burning or charring, for example.

In some embodiments, the cautery device housing 190 may be configured with a replaceable power source, thus, the cautery device housing 190 may be reused in subsequent operations, while assuring the power supply is adequate for the subsequent operations.

In some embodiments, the power switch 191 may be configured to complete the circuit between the power source, the electrodes 130, and the at least one wire 110, causing the at least one wire to heat to the designated temperature. As noted herein, in some embodiments, the cautery tip may be designed to heat to a temperature of no more than 1300° F. A combination of the expected power from the power supply and the resistance level of the at least one wire 110 (e.g., due to its wire diameter and overall length) may be designed such that the temperature does not exceed 1300° F., thereby preventing burning, charring, and/or igniting of the medium being cut. In some embodiments, the medium may require a temperature above 1300° F. to form the cautery hole within a desired time, therefore in some embodiments, as discussed, the composition of the electrodes 130, the at least one wire 110 and/or the power source within the cautery device housing 190 may be customized to reach the requisite temperature to form the cautery hole within the medium, without burning, charring, and/or ignition of the medium. Such example embodiments may, for example, enable desirable cutting of medium other than grafts.

In some embodiments, the end stop 126 of the at least one shuttle 125 may abut the cautery device housing 190, such that the electrodes 130 are positioned within receiving holes within the cautery device housing 190. As mentioned above, the cautery device housing 190 may be configured for multiple uses. Thus, the cautery device housing 190 may be configured to remain sterile or be able to be sterilized. In some embodiments, the cautery device housing 190 may be maintained within a sterile bag. In some embodiments, the ends 131 of the electrodes 130 may be configured to pierce the sterile bag and be inserted into the openings within the cautery device housing 190.

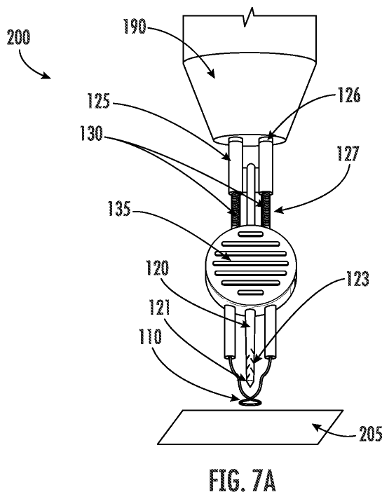

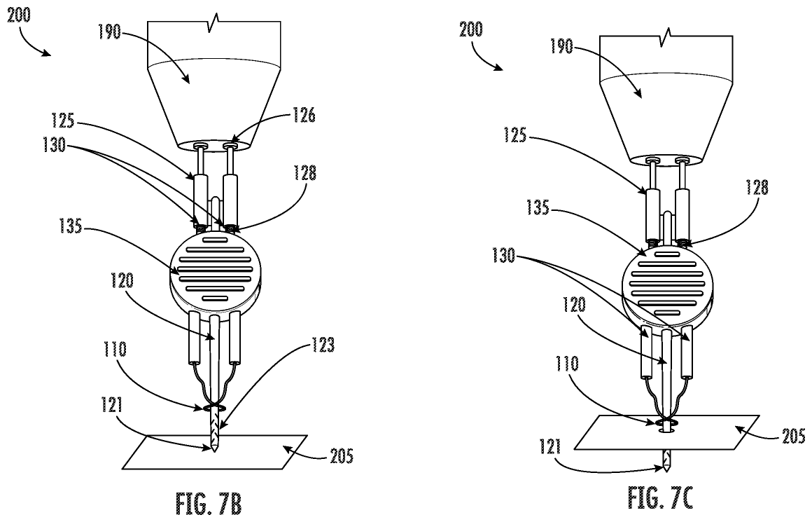

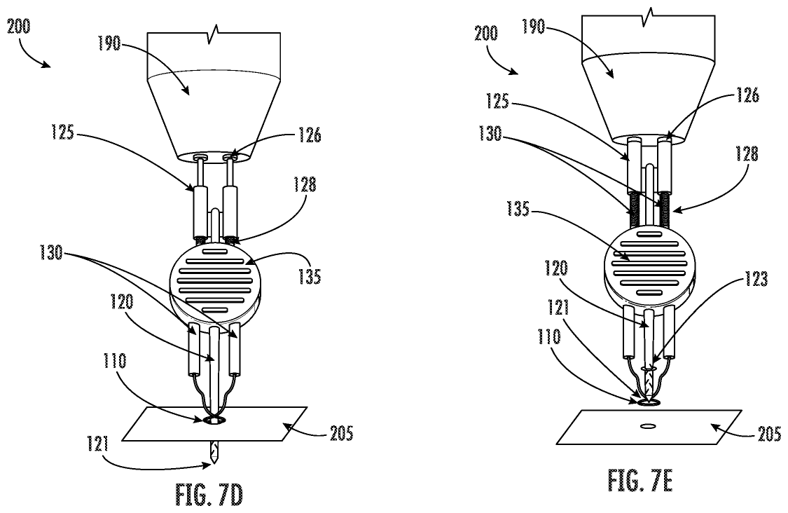

FIGS. 7A-E illustrate the cautery device 200 in use to create a hole within a medium, for example a graft 205. In some embodiments, before utilizing the cautery device 200, the graft 205 may be tensioned (e.g., pulled tight). FIG. 7A illustrates the tip portion 100 inserted into the cautery device housing 190 via the electrodes 130 such that the end stop 126 abuts the cautery device housing 190. Thus, when the power is activated, the tip portion 100 (e.g., the electrodes 130 and the at least one wire 110) may complete the circuit, allowing the at least one wire 110 to heat to the requisite temperature.

As discussed, the at least one biasing element 127 is configured to be biased in a decompressed position, such that the retrieval portion 120 is biased in the retracted position, as illustrated in FIG. 7A. In the retracted position, the retrieval portion 120 may not breach the cautery plane (e.g., 115, FIG. 5A) of the cautery shape formed by the at least one wire 110. In some embodiments, the tip 121 of the retrieval portion 120 may be positioned just above the cautery shape (e.g., between the stabilizer 135 and the cautery shape). In some embodiments, the at least one shuttle 125 may abut the end stop 126 in the retracted position, as illustrated.

With reference to FIG. 7B, the retrieval portion 120 may be moved into the extended position by moving the at least one shuttle 125, thereby compressing the at least one biasing element 127. In some embodiments, the retrieval portion 120 may be positioned in the extended position prior to contacting the graft 205, such as illustrated in FIG. 7B, while in other embodiments, the retrieval portion 120 may be configured to pierce the graft 205 when the retrieval portion 120 is moved to the extended position, as illustrated in FIG. 7C.

As illustrated, when the retrieval portion 120 is in the extended position, the retrieval portion 120 extends past the cautery plane defined by the at least one wire 110. In this regard, when the retrieval portion 120 and cautery device 200 are so moved, the tip 121 of the retrieval portion 120 may pierce through the graft 205. After piercing the graft 205, the retrieval portion 120 may be extended further, for example, by moving the entire cautery device 200, or in some embodiments, by extending the retrieval portion 120 via the at least one shuttle 125.

By moving the retrieval portion 120 through the graft 205, the barbs 123 may catch the graft 205, such as when the shuttle 125 is released and the bias of the biasing element 127 cause slight retraction of the retrieval portion 120. Once the graft 205 is retained by at least one of the barbs 123, the bias of the biasing element 127 may cause the desired portion of the graft 205 to pull back towards the at least one wire 110. In this regard, tension within the graft 205 allow the graft 205 to contact the at least one wire 110 and prevents the graft 205 from being pulled though the cautery shape by the at least one biasing element 127.

As mentioned, in some embodiments, the graft 205 may be slightly retracted into contact with the at least one wire 110. Once the graft 205 abuts the at least one wire 110, the power switch may be activated such that the at least one wire 110 heats up to cut (e.g., melt) the desired portion of the graft 205. As discussed, the at least one wire 110 is configured such that the at least one wire 110 may heat to a desired temperature (e.g., the melting point of the graft) without reaching an ignition temperature. This process is illustrated in FIG. 7D.

With reference to FIG. 7E, once a hole 206 is created within the graft 205, the at least one biasing element 127 retracts the retrieval portion 120 to the retracted position. In this regard, formation of the hole 206 alleviated tension within graft 205. Upon alleviation of the tension, the graft 205 is removed from contact with the tip portion 100 and thus, heat from the at least one wire 110 is removed, thereby preventing extended contact time between the graft 205 and the at least one wire 110 which may otherwise cause charring and/or burning of the graft 205. Said differently, the tip portion 100 is configured to cause tension within the graft 205 until the cautery hole 206 is formed, and since the tip portion 100 brought the graft 205 into contact with the at least one wire 110, after the cautery hole 206 is formed, the graft 205 is removed from contact with the at least one wire 110 instantaneously, thereby preventing the graft 205 from burning. Thus, the graft 205 comprises a cautery hole 206, and a portion 207 of the graft 205 corresponding to the cautery hole 206 is retained on the barbs 123 of the retrieval portion 120. Thus, the removal of the portion 207 of the graft 205 may be expedited as the portion 207 is retained when the cautery device 300 is moved away from the graft 205. Further, the location of the portion 207 is readily identifiable and the portion 207 is not able to fall into the graft, for example. In some embodiments, the cautery device 200 may be used to make multiple cautery holes 206 within the same operation, and the retrieval portion 120 may be configured to retain multiple portions 207 of the graft 205.

In some embodiments, the at least one wire 110 may be returned to contact with the graft 205 to adjust and/or expand the size of the cautery hole 206. In this regard, the at least one wire 110 may be positioned within the cautery hole 206 and moved about the perimeter of the cautery hole 206 thereby melting the graft 205 and increasing the size of the cautery hole 206. In this regard, even if the cautery tip may define a desirable shape, the cautery tip may still be used for customized shaping.

### Example Flowchart(s) and Operations

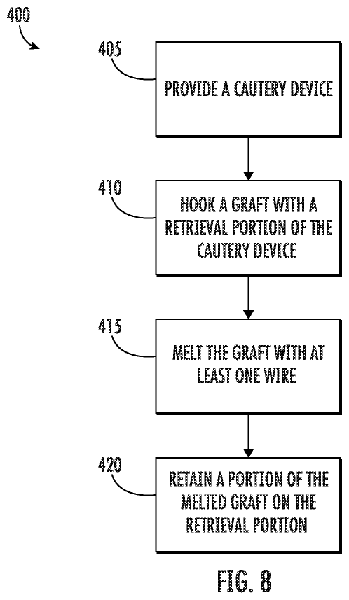

Various examples of the operations performed in accordance with embodiments of the present invention will now be provided with reference to FIG. 8.

FIG. 8 illustrates a flow chart according to an example method 400 of creating a hole (e.g., in a graft) using a cautery device. At operation 405, a cautery device is provided. In some embodiments, the cautery device may be cautery device 300 including the insert 100 and the cautery device housing 190. At operation 410, a graft may be punctured (e.g., hooked) with the retrieval portion (e.g., retrieval portion 120) of the cautery device. Tension in the surface of the graft and counterforce from the at least one wire now in contact with the surface may hold the retrieval portion against the bias of the biasing element. In some embodiments, at operation 415, electricity may be provided to at least one wire to cause the wire to heat so as to cut (e.g., melt) through the thickness of the graft, such as in the shape of the wire in contact with the graft. At operation 420, the force of the at least one biasing element may over come the tension of the graft after a hole is cut into the graft, and a portion of the graft may be retracted and retained on the retrieval portion. The tension within the graft may be alleviated and retract from the at least one wire. The electricity may cease, and the cautery device may be moved further from the graft, with the graft (or other surface) exhibiting a perfectly cut hole. Notably, the cut portion has been safely removed and retained (and will not fall into the surgical area). Optionally, in some embodiments, the tip portion may be used multiple times in the same operation, such that the graft may comprise multiple holes, and the needle may retain the corresponding graft portions stacked on the retrieval device.

### CONCLUSION

Many modification and other embodiments of the inventions set forth herein will come to mind to one skilled in the art to which these inventions pertain having the benefit of the teaching presented in the foregoing description and the associated drawings. Therefore, it is to be understood that the embodiments of the invention are not to be limited to the specific embodiments disclosed and that the modification and other embodiments are intended to be included within the scope of the invention. Moreover, although the foregoing descriptions and the associated drawings describe example embodiments in the context of certain example combinations of elements and/or functions may be provided by alternative embodiments without departing from the scope of invention. In this regard, for example, different combinations of elements and/or functions than those explicitly described above are also contemplated within the scope of the invention. Although specific terms are employed herein, they are used in a generic and descriptive sense only and not for purposes of limitation.

What is claimed is:

1. A tip portion for a cautery device defining a device axis, the tip portion comprising:
a first electrode and a second electrode, wherein the first electrode and the second electrode are spaced apart; and
at least one wire comprising:
a first end in electrical connection with the first electrode;
a second end in electrical connection with the second electrode; and
a central portion in electrical communication with the first electrode and the second electrode, wherein the central portion defines a cautery shape, wherein the cautery shape comprises a continuous perimeter extending in a cautery plane, and wherein the cautery plane is angled relative to the device axis.

2. The tip portion of claim 1, further comprising a retrieval portion positioned between the first electrode and the second electrode.

3. The tip portion of claim 2, further comprising a stabilizer, wherein the first electrode, the second electrode and the needle extend through the stabilizer, and wherein the stabilizer is configured to maintain spacing between the first electrode and the second electrode.

4. The tip portion of claim 2, wherein the retrieval portion extends along and is moveable along a retrieval axis.

5. The tip portion of claim 4, further comprising at least one shuttle connected to the retrieval portion and moveable along the retrieval axis to enable movement of the retrieval portion between a retracted position and an extended position.

6. The tip portion of claim 5, wherein, when the retrieval portion is in the extended position, a tip of the retrieval portion extends past the cautery plane.

7. The tip portion of claim 6, wherein, when the retrieval portion is in the extended position, the tip of the retrieval portion extends through the continuous perimeter of the cautery shape.

8. The tip portion of claim 6, further comprising at least one biasing element configured to bias the retrieval portion to the retracted position.

9. The tip portion of claim 8, wherein when the retrieval portion extends through the cautery plane, the retrieval portion is configured to pass through a medium being cut, wherein the bias of the biasing element causes the retrieval portion to pull the medium toward the at least one wire.

10. The tip portion of claim 9, wherein, upon application of electricity to the at least one wire, the at least one wire causes cutting of a hole in the medium in the cautery plane, wherein, upon completion of the cutting of the hole, tension from the medium holding the retrieval portion against the bias of the biasing element is removed to enable the retrieval portion and a corresponding portion of the medium attached thereto to move to the retracted position such that the portion of the surface being cut is retained by the retrieval portion.

11. The tip portion of claim 2, wherein the retrieval portion is barbed.

12. The tip portion of claim 2, wherein the retrieval portion is a needle.

13. The tip portion of claim 2, wherein the retrieval portion is configured to provide suction.

14. The tip portion of claim 1, wherein the perimeter of the cautery shape defines a circle, oval, or diamond.

15. The tip portion of claim 1, wherein the first electrode and the second electrode are configured to cause the wire to heat to a temperature between 700 F-1300 F.

16. The tip portion of claim 15, wherein the temperature is below a burning temperature of a vascular graft.

17. The tip portion of claim 1, wherein the cautery plane is perpendicular relative to the device axis.

18. The tip portion of claim 1, wherein the cautery plane extends at an angle within a range of 450-900 relative to the device axis.

19. A cautery tip portion for a cautery device defining a device axis, the tip portion comprising:
a first electrode and a second electrode, wherein the first electrode and the second electrode are spaced apart;
at least one wire, wherein the at least one wire is in electrical connection with the first electrode and the second electrode, wherein the at least one wire defines a cautery shape, wherein the cautery shape defines an at least near-continuous perimeter in a cautery plane; and
a retrieval portion disposed between the first electrode and the second electrode, wherein the retrieval portion extends along and is moveable along a retrieval axis.

20. The cautery tip portion of claim 19, further comprising at least one shuttle connected to the retrieval portion and moveable along the retrieval axis to enable movement of the retrieval portion between a retracted position and an extended position.

21. The cautery tip portion of claim 20, wherein in the extended position, a tip of the retrieval portion extends past the cautery plane.

22. The cautery tip portion of claim 21, further comprising at least one biasing element, wherein the at least one biasing element is configured to bias the retrieval portion to the retracted position.

23. The cautery tip portion of claim 19, wherein the retrieval portion is barbed.

24. The cautery tip portion of claim 19, wherein the retrieval portion is a needle.

25. A method of creating a hole using a cautery device, the method comprising:
providing a cautery device defining a device axis, comprising a cautery tip portion, the cautery tip portion comprising:
a first electrode and a second electrode extending through a stabilizer, wherein the stabilizer is configured to maintain spacing between the first electrode and the second electrode;
at least one wire in electrical connection with the first electrode and the second electrode, wherein the at least one wire is configured into a cautery shape, wherein the cautery shape comprises an at least near-continuous perimeter extending within a cautery plane;
a retrieval portion disposed between the first electrode and the second electrode, wherein the retrieval portion extends along and is moveable along a retrieval axis;
at least one shuttle connected to the retrieval portion and moveable along the retrieval axis to enable movement of the retrieval portion between a retracted position and an extended position; and
at least one biasing element configured to bias the retrieval portion in the retracted position;
puncturing a graft with the retrieval portion, such that the graft is in contact with the at least one wire, wherein the retrieval portion provides tension on the graft;
cutting the graft in the cautery shape, wherein the first electrode and the second electrode are configured to heat the at least one wire to a temperature, wherein the temperature is configured to cut the graft; and
retaining a cautery shaped portion of the graft via the retrieval portion after the graft is cut.
